(19) United States
(12) Patent Application Publication
(10) Pub. No.: US 2012/0301914 A1
(43) Pub. Date: Nov. 29, 2012

(54) High Resolution Label-Free Sensor

(76) Inventors: John Stephen Peanasky, Big Flats, NY (US); Vitor Marino Schneider, Painted Post, NY (US); Elizabeth Tran, Painted Post, NY (US); Qi Wu, Painted Post, NY (US)

(21) Appl. No.: 13/480,523

(22) Filed: May 25, 2012

Related U.S. Application Data
(60) Provisional application No. 61/490,462, filed on May 26, 2011.

(51) Int. Cl. G02B 6/00; C12Q 1/02; G01N 21/17

(52) U.S. Cl. 435/29; 385/12; 422/82.11

(57) ABSTRACT

An optical sensor for label-independent detection, having improved spatial resolution and reduced angular sensitivity, the sensor including:
a substrate;
a waveguide grating adjacent the substrate; and
a waveguide coat layer adjacent or over the waveguide grating,
   the waveguide coat layer having a thickness (W) of from 30 nm to 300 nm, the waveguide grating having a teeth height (H) of from 0.2×W to 1×W, and for example, a waveguide core thickness (W_{core}=W−H) from 5 nm to 50 nm.

Also disclosed is a well-plate article, a well-plate reader system, and methods of using the well-plate and sensor articles, as defined herein.

This application claims the benefit of priority under 35 U.S.C. §119 of U.S. Provisional Application Ser. No. 61/490,462, filed on May 26, 2011, the content of which is relied upon and incorporated herein by reference in its entirety.

### BACKGROUND

The disclosure generally relates to a high resolution sensor article, and to a well plate article incorporating the high resolution sensor article, for use, for example, in label-free sensing.

### SUMMARY

The disclosure provides a high resolution sensor article, a well plate article incorporating the high resolution sensor article, and methods for making and using the articles.

### BRIEF DESCRIPTION OF THE DRAWING(S)

In embodiments of the disclosure:

FIG. 1 provides a general schematic of a sensor plate.

FIGS. 2a and 2b show grating profiles for a commercial Epic® grating sensor (FIG. 2a), and the disclosed high spatial resolution and high image sensitivity grating sensor (FIG. 2b).

FIG. 3 shows finite-difference time-domain (FDTD) simulations for a comparative commercially available sensor having an electric field on the grating surface and having various beam widths.

FIG. 4 shows analogous FDTD simulations according to FIG. 3 for the disclosed high spatial resolution and high image sensitivity sensor.

FIG. 5 shows the total power transmission (T region) and the reflection (R region) for various grating depths or teeth heights (H).

FIGS. 6A and 6B provide compilations of various gain beam outputs for teeth height (H) of 146 nm (‘+’), 120 nm (‘*’), 100 nm (‘o’) and 50 nm (‘x’) parameters.

FIG. 7 compares the angular alignment sensitivity of the commercially available (e.g., Epic®) grating teeth height (H) of 50 nm and the angular alignment sensitivity of the disclosed plate having a grating teeth height (H) of 120 nm.

FIG. 8 shows the expected exponential decay (1/e) of the simulated electrical field inside the sensing material.

FIG. 9 shows the expected bulk sensitivity (nm/index unit) normalized per wavelength shift of peak reflection for a change in refractive index in a solution.

FIG. 10 shows the expected surface sensitivity (nm/nm) normalized per wavelength shift of peak reflection for the presence of a 5 nm layer of biological material having an index n=1.5 on the surface of the sensor.

FIGS. 11a and 11b schematically show two options for fabricating the disclosed high spatial resolution and high image sensitivity sensor plates.

FIG. 12 shows representative series of microscope images of A549 cells after 24 h of culture on disclosed biosensors having different grating depths.

FIG. 13 shows two microscope images of sensor surfaces of a comparative commercial plate (left), and the disclosed sensor plate (right), each surface having A549 cells, seeded at 250 k cells/well, and the cells being aligned perpendicular to the grating direction.

FIG. 14 shows a series of microscope images of surfaces of the disclosed sensor, each surface having A431 cells after 24 h of culture on biosensors having various grating teeth depths.

FIG. 15 shows microscope images of a comparative commercial plate (left side) and a disclosed plate (right side), each having A431 cells, seeded at 5 k cells/well and 250 k cells/well, and aligned with the grating direction for the disclosed sensors.

FIG. 16 shows microscope images of THP-1 cells 2h after plating on biosensors having various grating depths.

FIG. 17 shows an analysis method for a typical cell assay having low medium and high cell counts.

FIG. 18 shows an example of the pixel selection method of responders based on time domain information.

FIG. 19 shows cumulative time traces (gray) and averaged traces (three single black lines) for selected responders.

FIG. 20 shows a comparison of average traces for THP-1 cell with medium cell concentration (5 k cells) for the several different plates having various grating teeth depths

FIG. 21 shows bar chart statistics of results for measuring wavelength shifts upon contact with a drug compound tests with A431 cells that show average wavelength shifts in picometers for selected plates.

FIG. 22 shows bar chart statistics of results for tests as in FIG. 21 with a different drug compound with THP-1 cells showing average shifts for selected plates

FIGS. 23a and 23b show microscopic images of the power reflectivity of, respectively, the comparative commercial standard plate (FIG. 23a) compared to the disclosed plate (FIG. 23b).

### DETAILED DESCRIPTION

Various embodiments of the disclosure will be described in detail with reference to drawings. Reference to various embodiments does not limit the scope of the invention, which is limited only by the scope of the attached claims. Additionally, any examples set forth in this specification are not limiting and merely set forth some of the many possible embodiments of the claimed invention.

### DEFINITIONS

“Biosensor,” “sensor,” or like term refers to an article, that in combination with appropriate apparatus, can detect a desired analyte or condition. A biosensor combines a biological component with a physicochemical detector component. A biosensor can typically consist of three parts: a biological component or element (such as tissue, microorganism, pathogen, cells, cell component, a receptor, and like entities, or combinations thereof), a detector element (operating in a physicochemical way such as optical, piezoelectric, electrochemical, thermometric, magnetic, or like manner), and a transducer associated with both components. In embodiments, the biosensor can convert a molecular recognition, molecular interaction, molecular stimulation, or like event occurring in a surface bound cell component or cell, such as a protein or receptor, into a detectable and quantifiable signal. A biosensor as used herein can include liquid handling systems which are static, dynamic, or a combination thereof. In embodiments of the disclosure, one or more biosensor can be incorporated into a micro-article. Biosensors are useful tools and some exemplary uses and configurations are disclosed, for example, in PCT Application No. PCT/US2006/013539 (Pub. No. WO 2006/108183), published Dec. 10, 2006, to Fang, Y., et al., entitled “Label-Free Biosensors and Cells,” and U.S. Pat. No. 7,175,980. Biosensor-based cell assays having penetration depths, detection zones, or sensing volumes have been described, see for example, Fang, Y., et al. “Resonant waveguide grating biosensor for living cell sensing,” Biophys. J., 91, 1925-1940 (2006). Microfluidic articles are also useful tools and some exemplary uses, configurations, and methods of manufacture are disclosed, for example, in U.S. Pat. Nos. 6,677,131, and 7,007,709. U.S. Patent Publication 2007/0141231 and U.S. Pat. No. 7,175,980, disclose a microplate assembly and method. These documents are hereby incorporated by reference in their entirety.

The articles and methods of the disclosure are particularly well suited for biosensors based on label-independent detection (LID), such as for example an Epic® system or those based on surface plasmon resonance (SPR). The articles, and methods of the disclosure are also compatible with an alternative LID sensor, such as Dual Polarized Intereferometry (DPI). In embodiments, the biosensor system can comprise, for example, a swept wavelength optical interrogation imaging system for a resonant waveguide grating biosensor, an angular interrogation system for a resonant waveguide grating biosensor, a spatially scanned wavelength interrogation system, surface plasmon resonance system, surface plasmon resonance imaging, or a combination thereof.

Commonly owned and assigned copending U.S. Patent Application Publication 2007/0154356 (U.S. Ser. No. 11/436,923) discloses at para. [0042] an optically readable microplate having an attached mask with apertures.

“About” modifying, for example, the quantity, dimension, process temperature, process time, and like values, and ranges thereof, employed in describing the embodiments of the disclosure, refers to variation in the numerical quantity that can occur, for example: through typical measuring and handling procedures used; through inadvertent error in these procedures; through differences in the manufacture, source, or quality of components and like considerations. The term “about” also encompasses amounts that differ due to aging of or environmental effects on components. The claims appended hereto include equivalents of these “about” quantities.

“Optional,” “optionally,” or like terms refer to the subsequently described event or circumstance can or cannot occur, and that the description includes instances where the event or circumstance occurs and instances where it does not. For example, the phrase “optional component” or like phrase means that the component can or can not be present and that the disclosure includes both embodiments including and excluding the component.

“Consisting essentially of” in embodiments refers, for example, to a sensor article, to a microplate including at least one sensor article, to optical readers and associated components, to an assay, to method of using the assay to screen compounds, and to articles, devices, or any apparatus of the disclosure, and can include the components or steps listed in the claim, plus other components or steps that do not materially affect the basic and novel properties of the articles, apparatus, or methods of making and use of the disclosure, such as particular components, a particular light source or wavelength, a particular surface modifier or condition, or like structure, material, or process variable selected. Items that may materially affect the basic properties of the components or steps of the disclosure, or that may impart undesirable characteristics to aspects of the disclosure include, for example, an optical sensor article having a disfavored large W_{core} value, as defined herein, compared to the disclosed optical sensor article.

The indefinite article “a” or “an” and its corresponding definite article “the” as used herein means at least one, or one or more, unless specified otherwise.

Abbreviations, which are well known to one of ordinary skill in the art, may be used (e.g., “h” or “hr” for hour or hours, and “rt” for room temperature, “nm” for nanometers, and like abbreviations).

Specific and preferred values disclosed for components, times, operations, and like aspects, and ranges thereof, are for illustration only; they do not exclude other defined values or other values within defined ranges. The article, apparatus, and methods of the disclosure include those having any value or any combination of the values, specific values, more specific values, and preferred values described herein.

Label-free imaging methods continue to evolve and can now provide spatially resolved high content label free responses within each sensor (see for example, commonly owned and assigned U.S. Pat. No. 7,599,055, to Gollier et al., entitled “Swept wavelength imaging optical interrogation system and method for using same”). The Corning, Inc., Epic® system is a high throughput label free detection technology platform for studying surface phenomena of, for example, bio-molecular interactions and live cells. A commercially available Epic® instrument can detect the average response of each biosensor in a microplate. The commercially available Epic® platform has two distinct parts: a reader and a sensor plate. The reader sends to the sensor plate, for example, a broadband light with a certain spectral content and receives a narrowband light as its input. The analysis of, for example, the wavelength shift in the narrowband light can provide information regarding chemical binding or biological activity. The sensor plate contains a grating-type resonator and the light from the reader interrogates the plate. The resonator design maximizes reflection and sensitivity simultaneously, while maintaining alignment tolerances at a manageable level from the perspective of mechanical design. The light reflected from the sensor plate is narrowband and the grooves of the grating can also enable the spatially selective attachment of biological materials, such as cells.

In embodiments, the present disclosure provides a sensor, and a well plate incorporating the sensor, which sensor is particularly useful in, for example, a 2D imaging-based Epic® reader (see, U.S. Pat. No. 7,599,055, supra., and commonly owned and assigned copending patent application, now U.S. Ser. No. 13/021,945, to Q. Wu, entitled “High resolution label free imaging,” first filed Feb. 22, 2010).

In embodiments, the disclosure provides a sensor article and method of use of the sensor article having improved spatial resolution of the optical sensor to, for example, less than about 100 micrometers, such as from 1 to 10 micrometers. In embodiments, the disclosure provides an sensor article, a well plate article incorporating the sensor article, and a method of using the articles having high spatial resolution and high image sensitivity for biochemical, live-cell, and like label-independent-detection (LID) assays.

In embodiments, the present disclosure provides a sensor plate having, for example:

improved spatial resolution, for example, allowing an operator to visualize, for example, single cells, such as individual cells, a small group of cells, or a cluster of cells; and

improved reflectivity of the signal while retaining high spatial resolution and high sensitivity without degrading other aspects of the design, such as alignment (angular) tolerances and overall surface and bulk sensitivity.

In embodiments, the present disclosure provides a sensor and sensor plate for use in, for example, a point based reader, a 2D imaging-based Epic® reader, and like readers.

In embodiments, the disclosure provides a sensor article, a microplate article incorporating one or more of the sensor articles, a method of making and characterizing the sensor and microplate articles, and methods of using the sensor and microplate in high spatial resolution and high image sensitivity label-free assays for chemical, biochemical, and cellular applications.

In embodiments, the disclosure provides an optical sensor comprising:

a substrate;

a waveguide grating adjacent the substrate; and

a waveguide coat or coating layer adjacent the waveguide grating, the waveguide coat or coating layer having a thickness (W) of from 30 nm to 300 nm, and the waveguide grating having a tooth or teeth height (H) of from 0.2×W to 1×W.

The waveguide coat layer can have a thickness (W) of, for example, from about 135 nm to about 160 nm, including intermedate values and ranges, and the waveguide grating teeth can have a height (H) of, for example, from 100 nm to 150 nm, and preferably from 110 nm to 125 nm, including intermediate values and ranges. The waveguide core (W_{core}=W−H) can have a thickness of, for example, from 5 nm to 50 nm, including intermediate values and ranges.

The spatial resolution of the disclosed sensor can be increased by, for example, from about 2 to about 3 times, including intermediate values and ranges. The image sensitivity of the disclosed sensor can be increased by, for example, from 2 to 2.5 times, including intermediate values and ranges. The angular sensitivity of the disclosed sensor, the well plate, and the reader acting on the well plate, can be decreased by, for example, from 1.1 to 2.5 times compared to a comparable system having a sensor, well plate, and reader combination having sensor waveguide grating teeth height (H) of 50 nm.

The common waveguide core thickness of consecutive grating teeth (W_{core}=W−H), that connects the grating W and H dimensions can be, for example, between 0 nm to 110 nm. When W_{core}=0 it leads to a device where the teeth height (H) of consecutive teeth barely touch edge-to-edge. The common waveguide core thickness of consecutive grating teeth, W_{core}=(W−H), that connects the grating W and H dimensions can be, for example, from −50 nm to 50 nm. Negative values for W_{core}, that is less than zero, indicate that the teeth height (H) of consecutive or adjacent teeth simply do not contact one another, and that they have a separation gap W_{core} between them.

In embodiments, the index of refraction of the disclosed waveguide material can be, for example, from 1.6 to 3.4, a preferred index of refraction from about 2.0 to about 2.4, a more preferred value for the index of refraction can be from about 2.2 to about 2.3, including intermediate values and ranges, and an even more preferred value for the index of refraction of the waveguide material can be, for example, 2.28, for example, when the waveguide coating is niobia and at 0.8 microns.

The substrate can be, for example, at least one of a polymer, such as PMMA, polyimides, or like polymeric materials, a composite, a metal, a glass, an inorganic oxide, an inorganic nitride, or a combination thereof, having, for example, an index of refraction from 1.3 to 2.2, and preferably an index of about 1.51 when the substrate comprises glass, or alternatively, a low-loss polymer having a similar index and having optical power attenuation at the wavelength of operation of, for example, less than or equal to 3 db/cm, and preferably less than 0.4 db/cm. The substrate, the waveguide grating, and the waveguide coat combination can have a relatively low-loss in the wavelength of operation and can have optical power attenuation at the wavelength of operation of less than or equal to 3 db/cm, and preferentially less than 0.4 db/cm. The “wavelength of operation” refers to the wavelength of the light from the light source used and the light measured where the sensor operates and presents a resonance peak that is detectable. The wavelength of operation can be, for example, from 200 nm to 2,000 nm, and preferably, in embodiments, from 700 nm to 900 nm, and in other embodiments, preferably from 800 to 840 nm, including intermediate values and ranges.

The substrate can be, for example, a glass, a plastic, or a combination thereof, the waveguide grating comprises a glass, and the waveguide coating comprising Ta₂O₅, Nb₂O₅, TiO₂, Al₂O₃, SiO₂, silicon nitride, or a mixture thereof, wherein the layer is adjacent to the surface of the glass. The sensor can further include, for example, at least one surface modifying chemical composition that is in contact with the waveguide coating, the composition having a thickness of from 30 nm to 150 nm. An exemplary surface modifying chemical composition, can be, for example, dEMA, or pre-blocked dEMA, as disclosed in commonly owned and assigned U.S. Pat. No. 7,781,203.

In embodiments, the sensor can be, for example, a biosensor, a chemo sensor, or a combination thereof. In embodiments, the sensor can be, for example, a resonant waveguide grating sensor.

In embodiments, the disclosure provides a system for label-free detection of an analyte in a microplate, the system comprising:

a light source for illuminating the at least one sensor of a microplate;

a receptacle to receive a microplate including at least one of the disclosed sensors; and

an imager to receive the optical image of the at least one sensor of the microplate.

The imager can have, for example, a pixel size of about 0.1 to 100 micrometers, and more preferrably 0.5 to 20 microns, and even more preferably of 3 to 12 micrometers, for example, when more economical system components are selected.

In embodiments, the disclosure provides a method of using the disclosed sensor comprising:

depositing at least one live-cell on the surface of the sensor; and

interrogating the sensor with a suitable reader having a suitable radiation source.

The disclosed sensor, plates, and method of use can visualize and measure single cells or small groups of cells by selecting a response of specific pixels via a threshold on the wavelength shift detected.

The at least one live-cell on the surface of at least one sensor can be, for example, a single cell, a single live-cell to about 1,000 live-cells, or from 2 to about 500 live-cells, including intermediate values and ranges.

The depositing at least one live-cell on the surface of the sensor can produce preferential alignment of the cells on the surface of the sensor with respect to the waveguide grating, waveguide grating coat, optional waveguide grating surface coat layer, or a combination thereof. The at least one live-cell on the surface of at least one sensor comprises a blood cell, a like small cells, e.g., 5 to 10 microns long dimension and 1 to 3 microns short dimension, and like small cell types and like aspect ratios, or a combination thereof. Blood cells can include, for example, red (erythrocytes), white (leukocytes), platelets (thrombocytes), or combinations thereof.

Compared to a commercially available standard 50 nm Epic® sensor plate from Corning, Inc. (see U.S. Pat. No. 7,599,055, supra.), the present disclosure provides several potential advantages and benefits including, for example:

an ability to visualize and measure a single cell or small cell arrangements, and to sense the cell's performance by high spatial resolution;

the cells being visualized can vary in size but even very small cells, such as ‘blood cell’ type, can be visualized;

the reflectivity for single cell or a small cell arrangements is considerably higher, e.g., by at least about 20% compared to a 50 nm plate, further enabling the measurement;

the tilt angle angular sensitivity of a well plate including the disclosed sensor is reduced by about 2 fold (e.g., new sensitivity of about 1.4) compared to a 50 nm plate (e.g., old sensitivity of about 0.6), leading to a mitigated plate that is easier to manufacture, easier to align in situ, and has relaxed mechanical operation criteria;

the bulk and surface sensitivities of the sensor for binding are excellent, and are comparable the sensitivities available with the commercially available Epic® system;

the process and material used in the making the sensor and plate are compatible with existing manufacturing capabilities and skill sets; and

the cost per unit of the disclosed sensor and well plate that incorporates the sensor is comparable to existing sensors and well plates.

These and other advantages are disclosed herein.

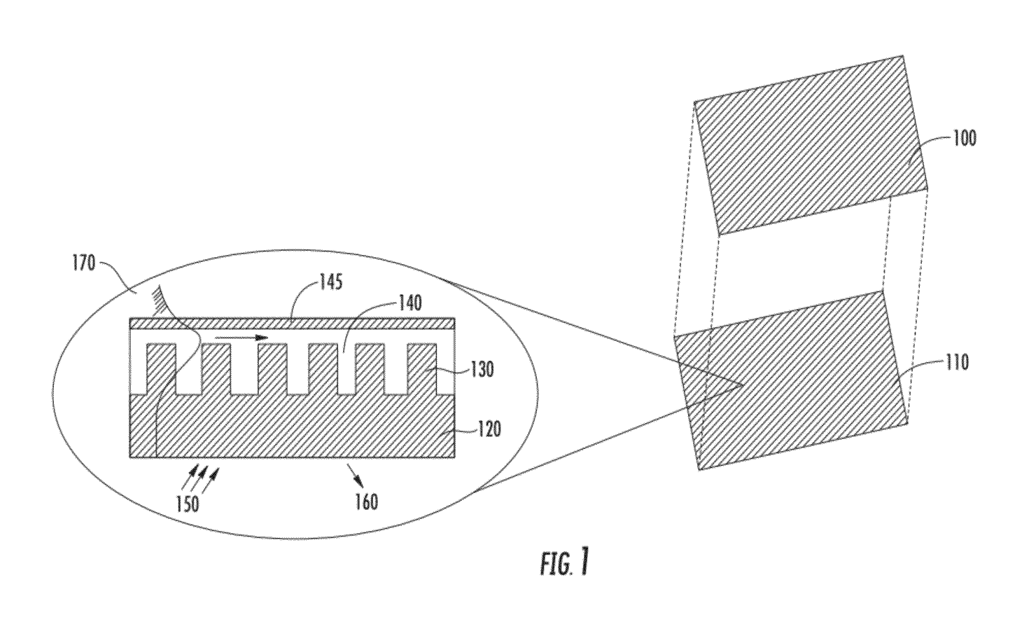

Referring to the Figures, FIG. 1 shows an exploded assembly of a sensor plate or well plate article generally having at least one sensor article within one or more wells including, for example, a microplate comprised of a body (100) and an insert (110). The body (100) provides structural integrity and wells to retain assay liquid. The insert (110) provides a bottom to individual well and provides a sensor comprising a substrate (120), having a waveguide (130), a waveguide surface coat (140), and optionally a thin surface coat (145) on the waveguide coating (140). The substrate (120) and waveguide (130) can be made of the same material or dissimilar materials.

A radiation source, such as a broadband source optionally having a collimating optics (not shown), provides an incident beam (150) to the sensor article, and results in a reflected beam (160) that includes sensor interrogation information arising from the evanescent wave (170). An image recorder (not shown) processes the reflected beam (160) and provides information regarding changes or shifts in wavelength. The incident beam can contact the bottom of the insert (110) at an angle or at normal incidence. The reflected narrowband light (160) that contains information regarding a possible binding event on the surface (front-side) of the sensor derived from a perturbation(s) in the evanescent wave (170) can result in a shift in the wavelength of the sensor's resonant peak. The plate having the sensor(s) can be inserted in a plastic case or body having multiple wells (“insert”) for rigidity and structural integrity. The completed assembled plate can then be combined with the reader to acquire data.

The radiation source can be, for example, a light emitting diode (LED), and like low- or non-coherent light sources. Other radiation sources can be selected if desired and properly adapted to the disclosed method. The radiation source can alternatively be or additionally include, for example, a fluorescent source capable of providing a fluorescent incident beam or fluorescence inducing incident beam.

In embodiments, the image recorder can be, for example, a CCD or CMOS camera, or like image recorder devices. A CCD having a very thin cover glass or no cover glass can provide improved image quality compared to a thick cover glass. The CCD or CMOS camera or like image recorder device can be, for example, free of a cover glass.

The optical sensor article can have a spatial resolution, for example, of from about 0.5 to about 1,000 micrometers, from about 1 to about 1,000 micrometers, from about 1 to about 100 micrometers, from about 1 to about 10 micrometers, and from about 5 to about 10 micrometers, including any intermediate ranges and values.

The sensor article can further include, for example, a microplate, a well plate, a microscope slide, a chip format, or like analyte container, support member, or sample presentation article, and optionally including, for example, microfluidic flow facility. In embodiments, the sensor article can have at least one microplate, having at least one well, the well having the at least one optical sensor therein, and the sensor can have a signal region and an optional reference region. The microplate can be an array of wells such as commerically available from Corning, Inc.

In embodiments, the incident beam can contact at least one sensor in, for example, at least one of: a single well, two or more wells, a plurality of wells, or all wells of the received microplate. In embodiments, an optical reader system having the incident beam can be selected and configured to interrogate an individual sensor, two or more sensors, such as in a row, column or cluster, or a full well-plate having a plurality of sensors. In embodiments, the reader can be configured so that the incident beam contacts, irradiates, or excites, one or more sensors, in one or more wells in sequential or systematic scanning fashion (see for example commonly owned and assigned copending application U.S. Ser. No. 61/231,446).

In embodiments, the microplate can have a base or substrate thickness, for example, of from about 10 micrometers to about 10,000 micrometers, about 50 micrometers to about 10,000 micrometers, and 100 micrometers to about 1,000 micrometers, and like values, including any intermediate values and ranges. A specific example of a microplate base thickness is, for example, of from about 0.1 millimeters to about 10 millimeters, such as 0.3 millimeters to about 1.0 millimeters. A thinner microplate base can, for example, reduce distortion and can improve image quality. A thin microplate base can be, for example, glass or like material having a thickness of about 0.7 mm to 1.0 mm and is representative of the thicknesses found in certain commercial products. Glass or like material having a thickness of less than about 0.4 mm is operatively a thin base plate material.

In embodiments, the disclosure provides a method of reading an evanescent wave sensor in the abovementioned reader having an engaged microplate having at least one of the disclosed sensors, comprising:

forming a microplate assembly by engaging the receptacle of the reader with a microplate having at least one well, the well having at least one sensor therein;

contacting the sensor at a first location with the incident beam; and

recording the image received from the contacted sensor with the image recorder.

The evanescent wave sensor can be, for example, a resonant waveguide biosensor, or like sensors, or a combination of such sensors.

The method can further comprise at least one relative moving (i.e., movement), of the microplate with respect to the incident beam to second location, and thereafter contacting at least one sensor of the microplate at the second location with the incident beam, and recording the image received with the image recorder. The relative movement of the microplate with respect to the incident beam can be accomplished by, for example, translating the beam stepwise, continuously, or a combination thereof, across the at least one sensor, similarly translating the sensor relative to the beam, or both.

In embodiments, the sensor can include on its surface, for example, at least one of a live-cell, a bioentity, a chemical compound, an selective reactive engineered coating, and like entities, or a combination thereof.

The spatial resolution of the recorded image can be, for example, from about 0.5 to about 10 micrometers, including intermediate values and ranges, and the excellent spatial resolution can be sufficient to accomplish, for example, sub-cellular label-free imaging, and like imaging objectives.

In embodiments, the method can, for example, further comprise simultaneously or sequentially contacting the sensor with a fluorescence inducing incident beam and recording the received fluorescent image with a suitable recorder. That is, to accomplish, for example, cellular or sub-cellular fluorescence imaging (see, for example, commonly owned and assigned copending application U.S. Ser. No. 12/151,179, US Pat. App. Pub. 2009/0325211, entitled “SYSTEM AND METHOD FOR DUAL-DETECTION OF A CELLULAR RESPONSE”).

In embodiments, the disclosure provides a method for enhancing the spatial resolution of resonant waveguide sensor comprising, for example:

interrogating at least one disclosed sensor article with a suitable reader; and

recording the image received from the interrogated sensor article with a suitable imager or like image recording device.

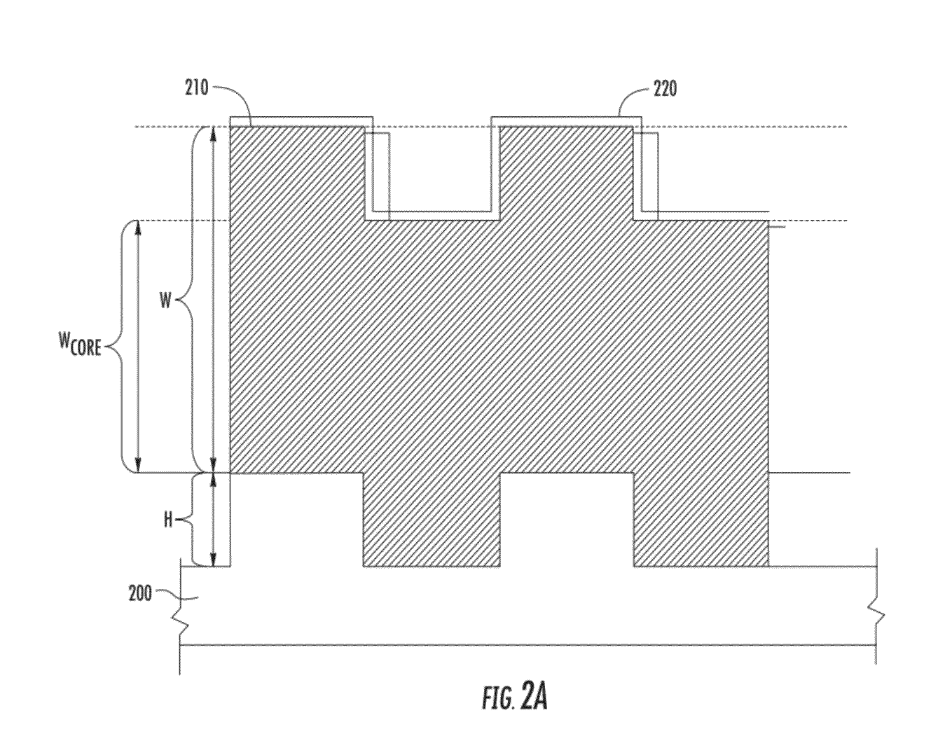

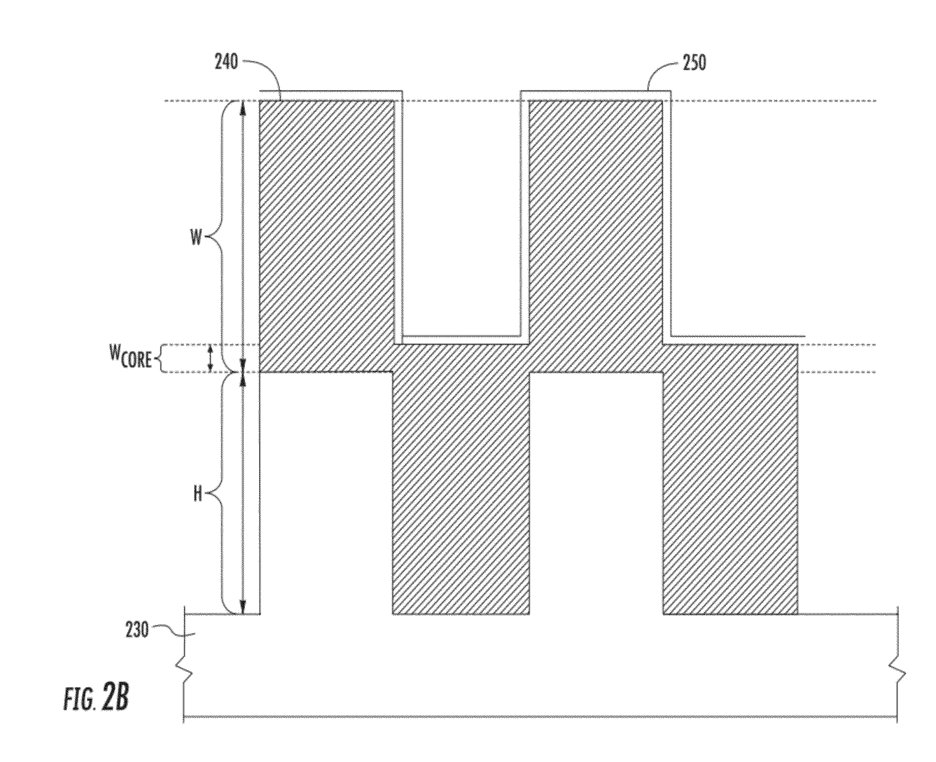

In embodiments, a significant aspect of the disclosure is to achieve and provide higher image resolution for a sensor plate in a 2D image reader. The technique used to achieve the higher image resolution property and result required an increase in the coupling coefficients for the light being coupled into the plate. This coupling coefficient is known to be a function of the intensity of the perturbations in the plate. After several trials and interactions, a solution was realized in a sensor plate design having significant improvement in image resolutions performance. A comparison of the disclosed inventive plate with the current commercial sensor plate is shown in FIG. 2. In embodiments, the disclosure provides a grating profile having high spatial resolution and high sensitivity compared to the commercial Epic® grating sensor. A commercial Epic® sensor (200) has grating teeth height dimensions (light region in FIG. 2a) (H) of 50 nm, a niobia coating (210) (dark region in FIG. 2a) W=146 nm, and a common region W_{core}=(W−H)=96 nm. An optional biological layer (220) having a thickness W_{bio} of about 5 nm can be included. The grating length can be from a few hundred to several thousand microns. For simulation purposes one grating length (L) used was 600 microns across. Here the indices of refraction were Niobia=2.285, N_{sub}=1.51 (glass or polymer), Nₛᵤₚ=1.333 (used as water), N_{bio}=1.5 (biological agent).

FIG. 2b shows an example of the disclosed high spatial resolution and high sensitivity sensor (230) having a waveguide grating teeth dimension (H) of 120 nm. The waveguide grating teeth dimension (H) can be, for example, from 80 nm up to the total thickness of the niobia coating. In this instance, the grating teeth dimension H was 120 nm, the niobia coating (240)(dark region) W was 146 nm, and has a common region W_{core}=(W−H)=26 nm. An optional biological layer (250) having a thickness W_{bio}=5 nm can be included. The grating length can be, for example, from a few hundreds to several thousands of microns. For simulation purposes one biological layer thickness dimension (L) used was 600 microns. The indices of refraction were N_{niobia}=2.285, N_{sub}=1.51 (glass or polymer), Nₛᵤₚ=1.333 (used as water), and N_{bio}=1.5 (biological agent). The overall thickness of the grating increased due to the larger grating tooth height dimension. However the overall thickness of the niobia coating remained the same, in this instance, 146 nm.

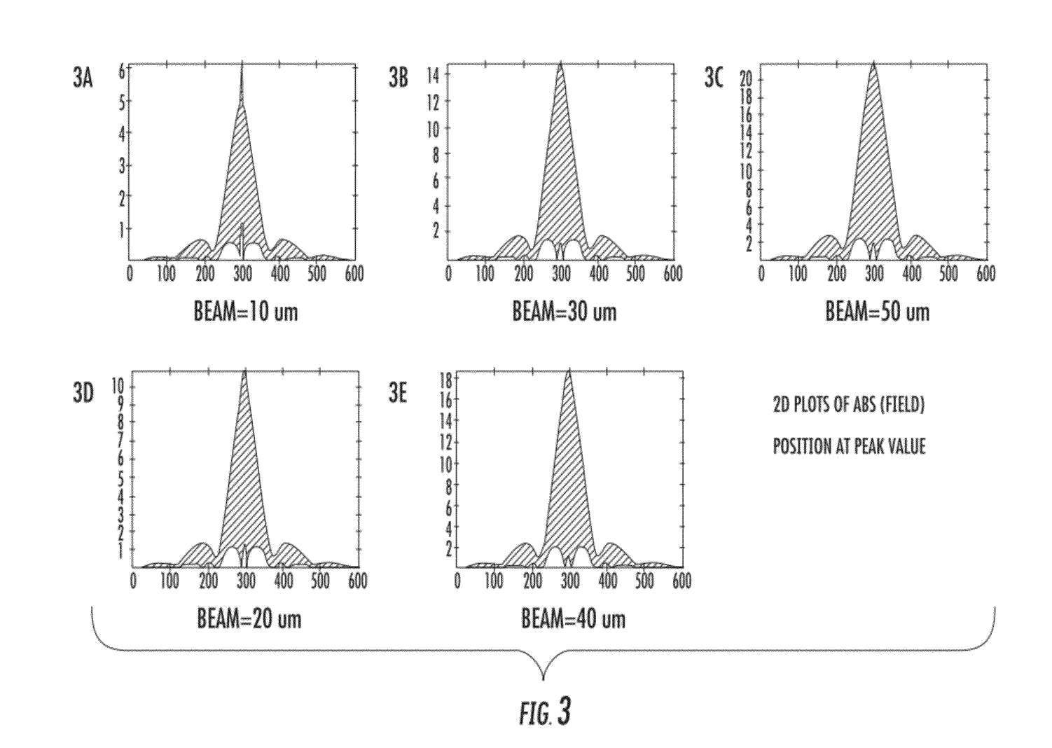

To understand how the disclosed sensor design changes the resolution and response of the sensor plate one can examine an Epic® sensor simulated by FDTD. FIG. 3 shows finite-difference time-domain (FDTD) simulations for a commercially available sensor (i.e., an Epic® sensor) having an electric field on the grating surface based on the incidence of a Gaussian beam having beam widths of 10 microns, 20 microns, 30 microns, 40 microns, and 50 microns, respectively. These several Gaussian beams were used to emulate the effect of the cell dimension on the grating surface. The electric field on the surface was then analyzed regarding its ‘effective width’ based on criteria of 3 dB, one sigma, two sigma, and three sigma of its peak value. These different thresholds for ‘effective width’ were used to emulate the possible different sensitivities of a camera based sensor. A commercially Epic® sensor having teeth dimensions (H) of 50 nm, niobia coating (dark region in graph) W of 146 nm, and a common region W_{core}=(W−H) of 96 nm. The biological layer thickness (W_{bio}) of 5 nm was assumed, and the grating length can be from a few hundred to several thousand microns. For simulation purposes one value used for the biological layer having thickness L was 600 microns. The indices of refraction were N_{niobia}=2.285, N_{sub}=1.51 (glass or polymer), Nₛᵤₚ=1.333 (used as water), and N_{bio}=1.5 (biological agent). The wavelength of light used in the simulations was λ□=0.8352 microns. In all instances the duty cycle was 50%.

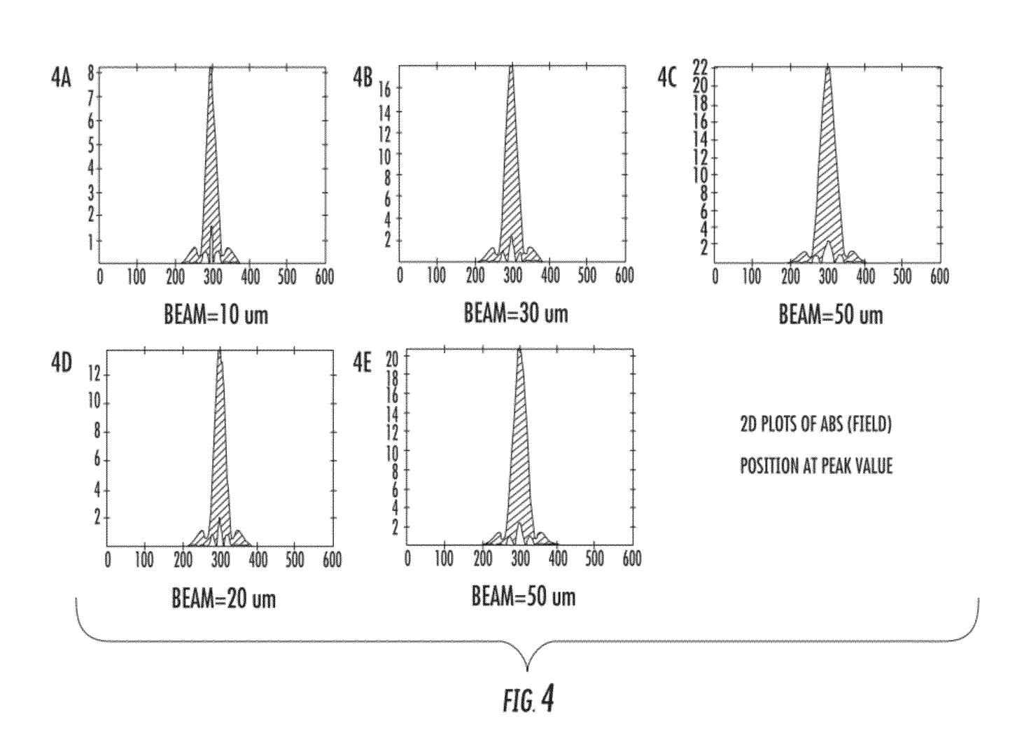

The FDTD simulations in FIG. 3 were then compared with actual experimental measurements shown in FIG. 4 for the disclosed high spatial resolution and high sensitivity sensor. Here again the electric field on the grating surface is computed based on the incidence of a Gaussian beam with widths of 10 um, 20 um, 30 um, 40 um and 50 um. These multiple Gaussian beams are used to emulate the effect of the cell dimension on the grating surface. The electric field on the surface is then analyzed regarding its ‘effective width’ based on criteria of 3 dB, one sigma, two sigma and three sigma of its peak value. These different thresholds for ‘effective width’ are used to emulate the possible different sensitivities of a camera based sensor. The current Epic sensor with teeth with dimensions H=120 nm, niobia coating (dark color in the graph) W=146 nm and a common region W_{core}=(W−H)=26 nm. Here, a biological layer with thickness W_{bio}=5 nm is assumed and the grating length can be from a few hundreds to several thousands of microns. For simulation purposes one used L=600 um. Here the indices of refraction are N_{niobia}=2.285, N_{sub}=1.51 (glass or polymer), Nₛᵤₚ=1.333 (used as water), N_{bio}=1.5 (biological agent). Wavelength used in simulations λ□=0.8352 microns. In all cases the duty cycle is 50%.

A comparison between FIG. 3 and FIG. 4 shows a significant reduction in the size of the electric field on the grating surface for the disclosed high resolution plates as well as higher power reflection.

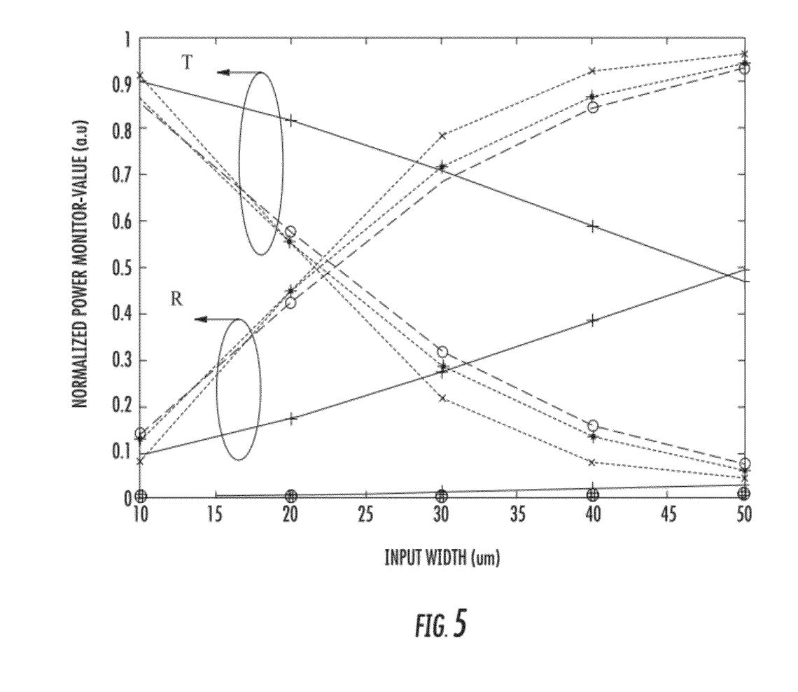

The overall power reflection changes for several different grating depths simulation can be observed in FIG. 5. Here, the total power transmission T and reflection R for grating depth H=146 nm (‘x’), 120 nm (‘*’), 100 nm (‘o’) and 50 nm (‘+’). Here the simulation is performed for the several Gaussian beam inputs from 10 microns to 50 microns. The total niobia coating thickness is W=146 nm and a common region W_{core}=(W−H), accordingly. The simulation was performed at its wavelength peak in each case. In all cases the grating length is 600 microns. Compared to the standard grating having height dimension H of 50 nm, a much larger reflectivity appears for any given Gaussian beam input width. This may be attributable to the larger coupling coefficients generated by the deeper grating depths. In all instances the duty cycle was 50%. Remaining power was lost at the edges of mathematical window and as shown by the dashed line near the x-axis baseline of the graph.

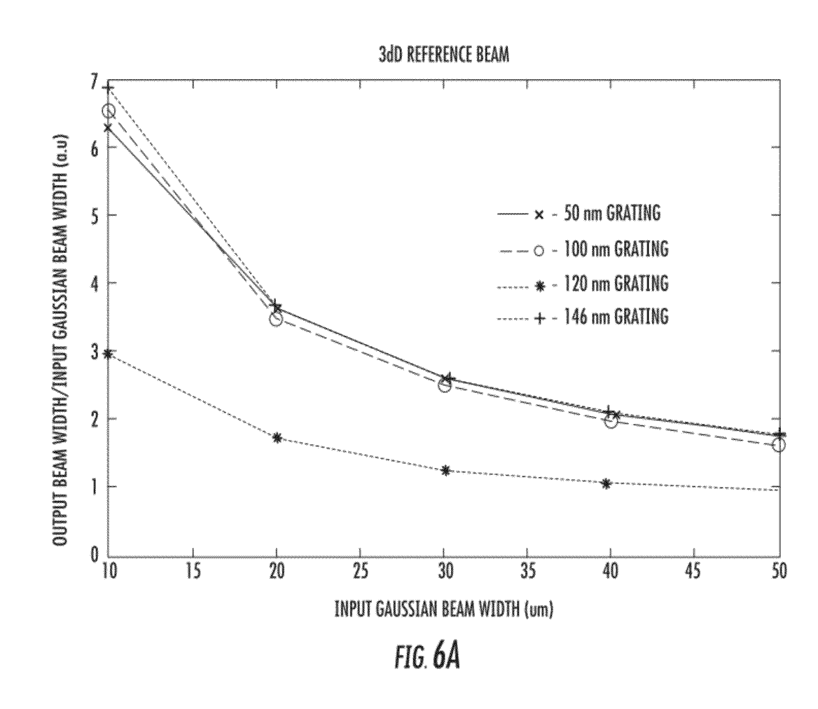

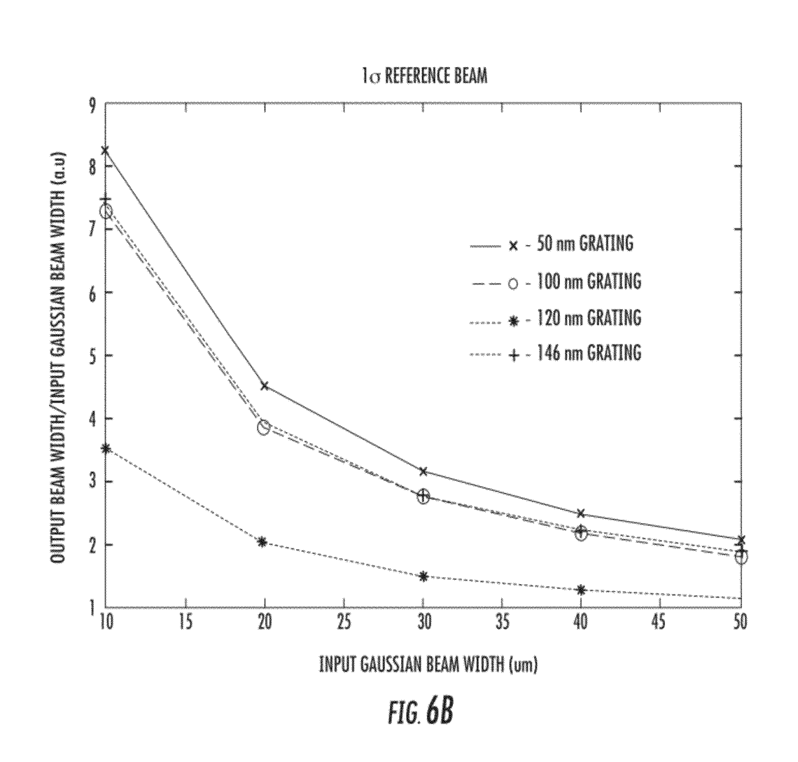

The relative reduction in beam size can be seen in the compilation provided in FIG. 6. FIG. 6 provides compilations of gain beam outputs for the teeth height (H) parameters. The ratio of the output beam in the top of the grating related to the initial Gaussian input beam was computed. The grating depths or heights (H) were 146 nm (‘+’), 120 nm (‘*’), 100 nm (‘o’), and 50 nm (‘x’), respectively. The grating teeth height (H) of 120 nm was the one height that seemed to provide the best performance to most thresholds in the reference. The grating teeth height (H) of 146 nm suffers from additional scattering due to the lack of a common waveguide ground. This lead to a larger beam spot size. The grating teeth height (H) of 120 nm appears to be a good compromise between a high coupling coefficient and lower scattering.

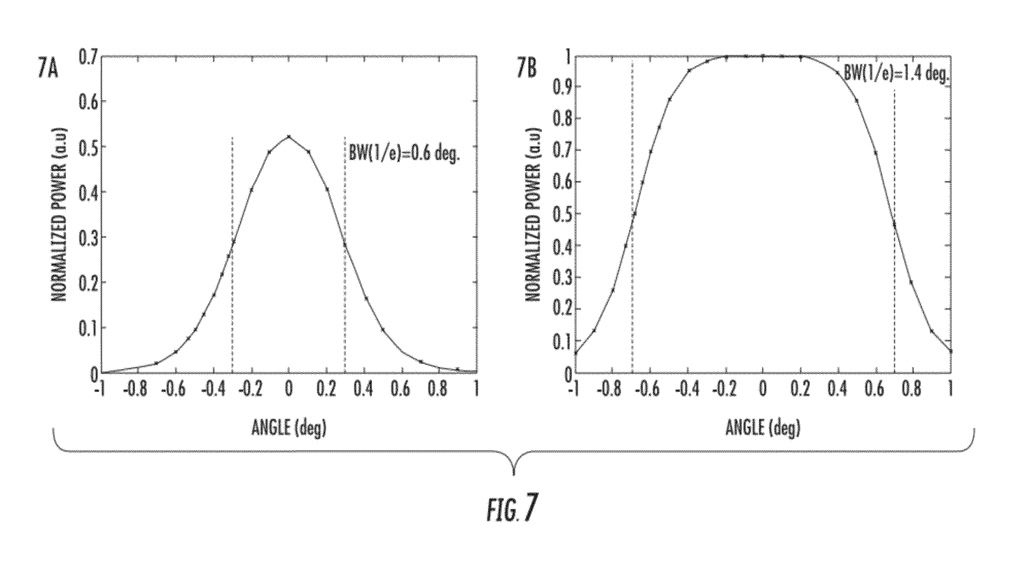

FIG. 7 compares the angular sensitivity to alignment for the commercially available (e.g., Epic®) grating teeth height (H) of 50 nm and the disclosed plate having a grating teeth height (H) of 120 nm. Details of each plate are similar to the ones described in FIG. 1. The disclosed high spatial resolution and high sensitivity plate had a larger angular tolerance compared to the comparative commercial plate, making the disclosed plate less sensitive to mechanical design issues of the optical reader.

In addition to higher resolution and higher reflectivity it is important to understand the effects of the disclosed plate on the overall angular sensitivity of the system. The angular sensitivity is described in FIG. 7 based on additional FDTD simulations at different angles of incidence. Here, one compares, the angular sensitivity to alignment for the current EPIC grating with H=50 nm and the propose grating with H=120 nm. Details of each plate are similar to the ones described in FIG. 2.

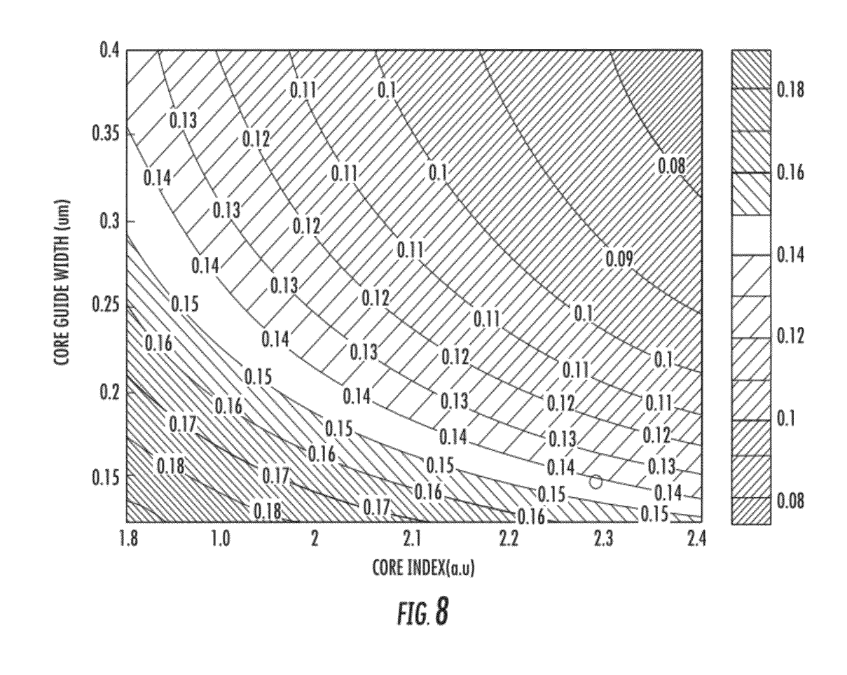

During the design process, care was taken to do not disturb the additional performance parameters of the current EPIC™ system. One critical parameter that is the penetration depth of the field can be observed in FIG. 8. Here, the expected exponential decay (1/e) of the electrical field inside the sensing material is computed. This would dictate approximately how deep one can probe inside the biological material. The white dot shows the location of the current designs irrespective of the value of teeth size H. This was intentionally done to maintain similar sensing depths between the comparative and the disclosed high spatial resolution and high sensitivity plates.

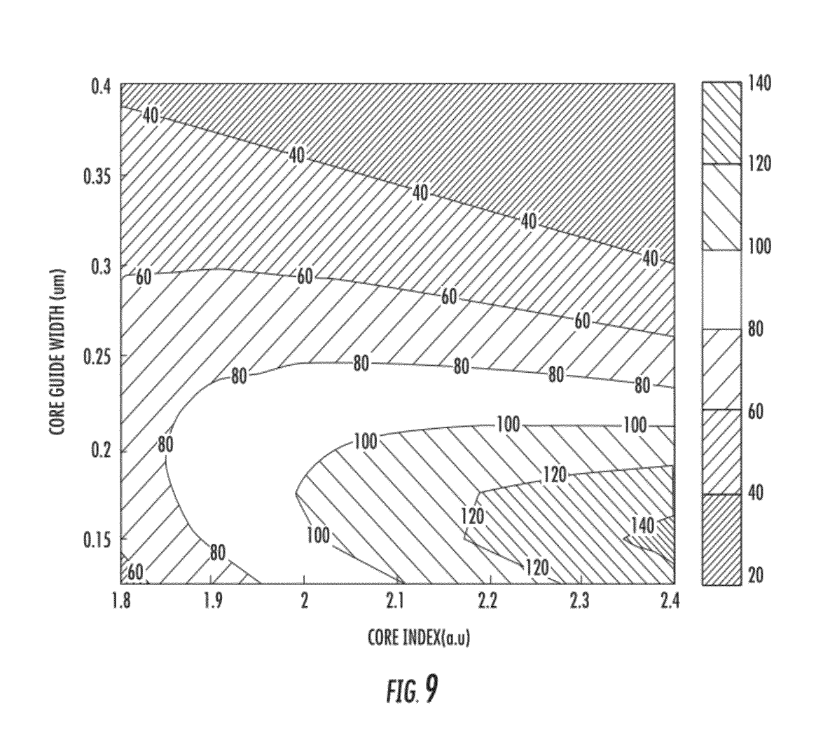

One additional parameter of interest is the bulk sensitivity described in FIG. 9. FIG. 9 shows the expected bulk sensitivity (nm/index unit) normalized per wavelength shift of peak reflection for a change in refractive index in a solution. The white dot (lower right) shows the location of the comparative commercially available design irrespective of the value of teeth size H. This was deliberately done to maintain similar bulk sensitivity between the comparative commercial plate and the disclosed high spatial resolution and high sensitivity plate.

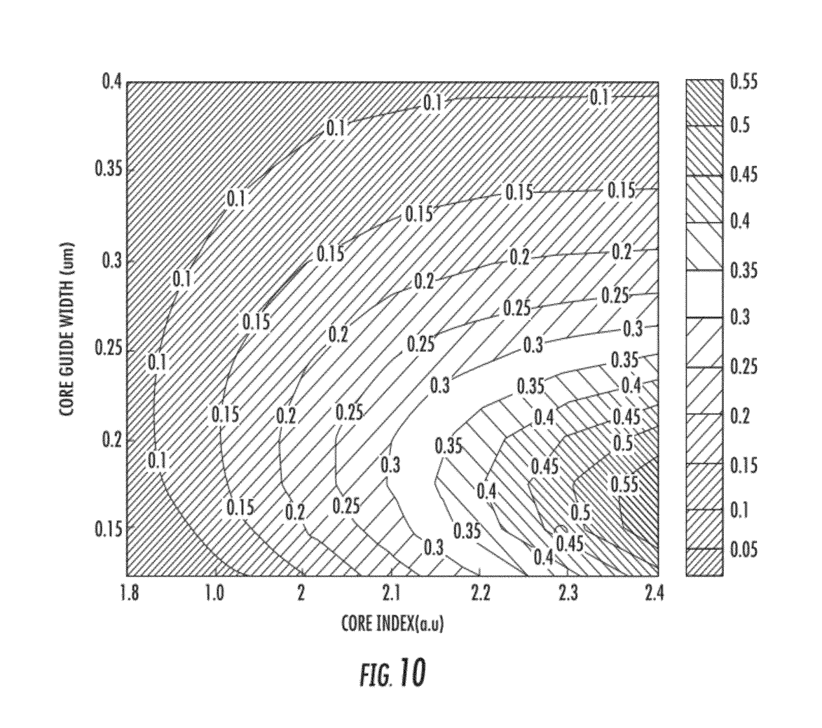

A second additional parameter of interest is the surface sensitivity described in FIG. 10. FIG. 10 shows the expected surface sensitivity (nm/nm) normalized per wavelength shift of peak reflection for the presence of a 5 nm layer of biological material having an index n=1.5 on the surface of the sensor. The white dot (lower right) shows the location of the comparative commercial design irrespective of the value of grating teeth size H. This was deliberately done to maintain similar surface sensitivity between the comparative commercial plate and the disclosed high spatial resolution and high sensitivity plate.

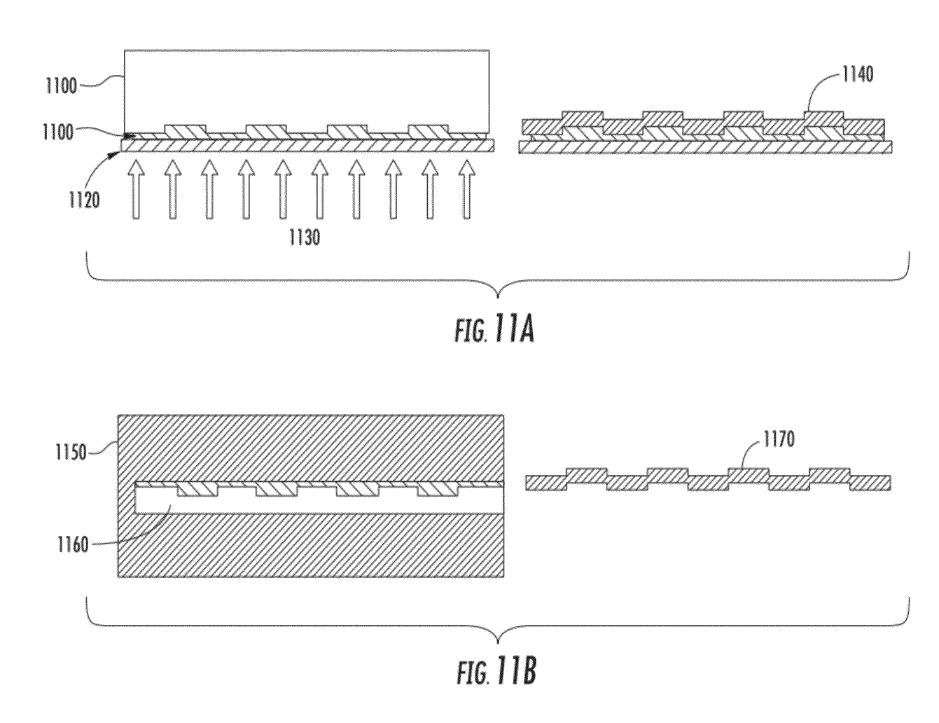

With all these parameters known and the design space mapped, device fabrication details of the disclosed high spatial resolution and high sensitivity plates were suggested and is exemplified in FIG. 11. FIGS. 11a and 11b shows two options for fabricating the disclosed high spatial resolution and high sensitivity sensor plates. FIG. 11a shows UV irradiation (1130) of a combined glass master stamp (1100) and UV curable resin (1110) on a glass substrate (1120) to form the sensor gratings followed by niobia coating (1140). FIG. 11b shows a less expensive thermoplastic mold based stamper (1150) acting on, for example, a thermoplastic polymer (1160), that after release from the stamper mold has a niobia coating (1170) deposited (e.g., low temperature PVD) to form the waveguide. Microplates used in the working examples of this disclosure were prepared by the UV irradiation process shown in and described for FIG. 11a. Five different glass master stamps with grating depths of, for example, 100 nm, 110 nm, 120 nm, 130 nm (v1), and 130 nm (v2) were prepared using a 193 nm lithographic stepper. The glass with the photoresist was then etched in a standard RIE etcher (Nextral) to create the master stamps in the depths indicated with a duty cycle of approximately 50% and pitch of 500 nm.

The glass master stamps were then used with the UV curable resin and replicated into several plates (25 plates) from which several were selected and used for niobia deposition tests. Plates were also prepared using this procedure to have a range of different grating depths (H) for testing with biological material and different cell based assays.

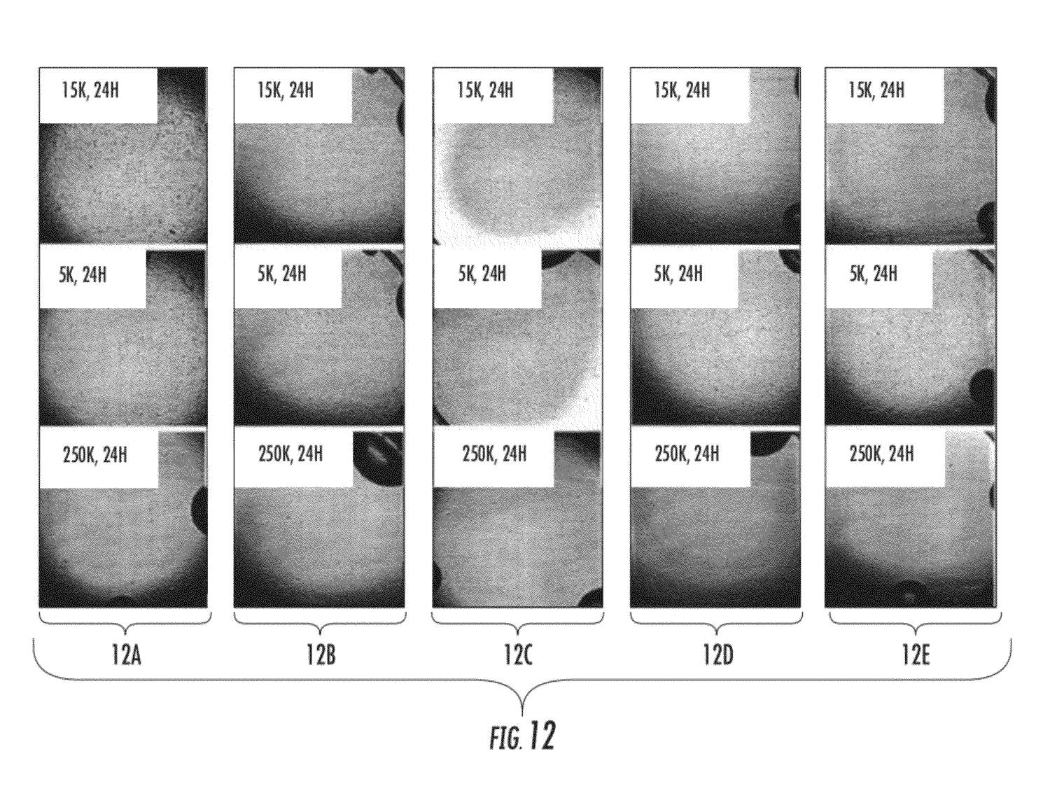

FIG. 12 shows five series (A through E left to right; ordered vertically at 15 k, 5 k, and 250 k cells/well) of microscope images of A549 cells after 24 h of culture on disclosed biosensors having different grating depths: A) 50 nm, B) 100 nm, C) 110 nm, D) 120 nm, and E) 130 nm. The A549 cells were seeded at 15 k, 5 k, and 250 k cells/well and aligned perpendicular to the grating of the commercial standard biosensors and the disclosed sensors. The results demonstrate good cell adherence properties for the disclosed sensors.

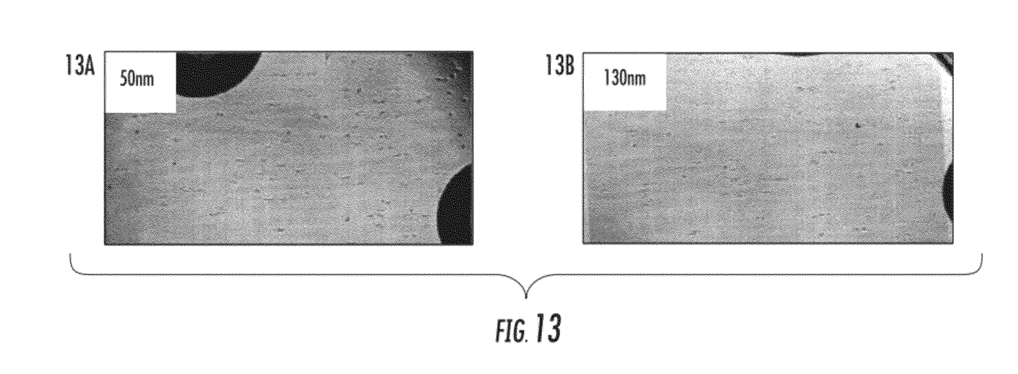

FIG. 13 shows microscope images of a comparative commercial plate (left) and a disclosed plate (right), each having A549 cells, seeded at 250 k cells/well, and aligned perpendicular to the grating direction.

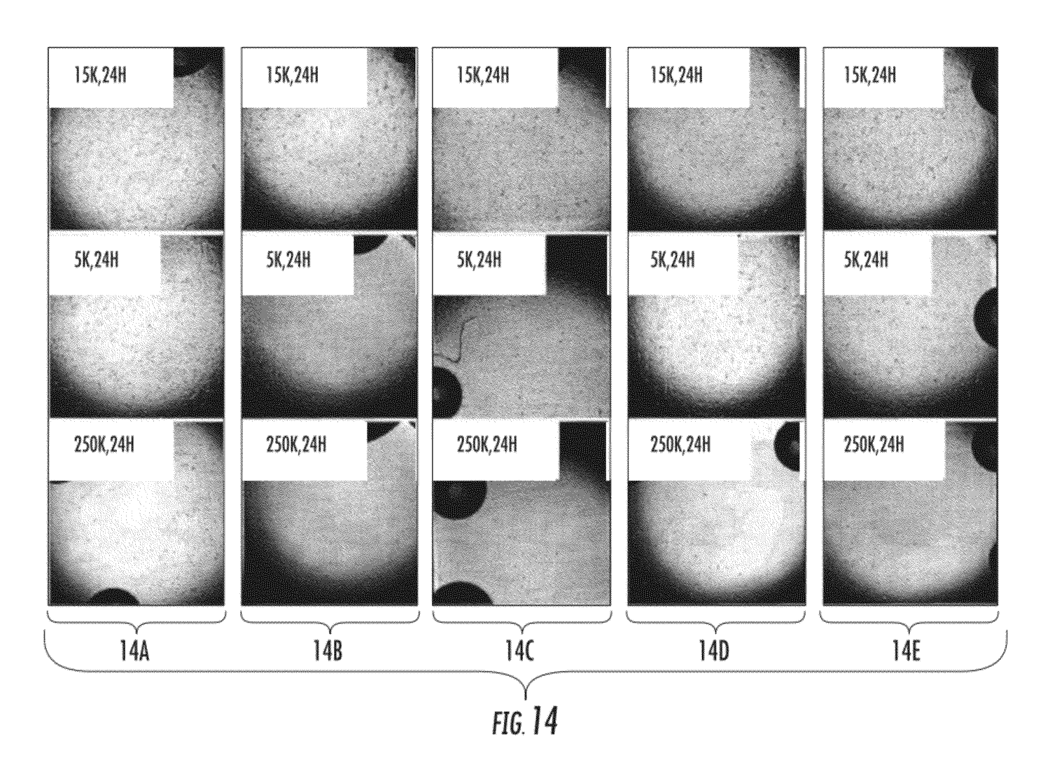

FIG. 14 shows five series (A through E left to right; ordered vertically at 15 k, 5 k, and 250 k cells/well) microscope images of A431 cells after 24 h of culture on biosensors having different grating teeth depths, respectively, of: A) 50 nm, B) 100 nm, C) 110 nm, D) 120 nm, and E) 130 nm. The A431 cells were seeded at 15 k, 5 k, and 250 k cells per well and were aligned parallel to the grating of the disclosed biosensors but not for the comparative commercial plate biosensors.

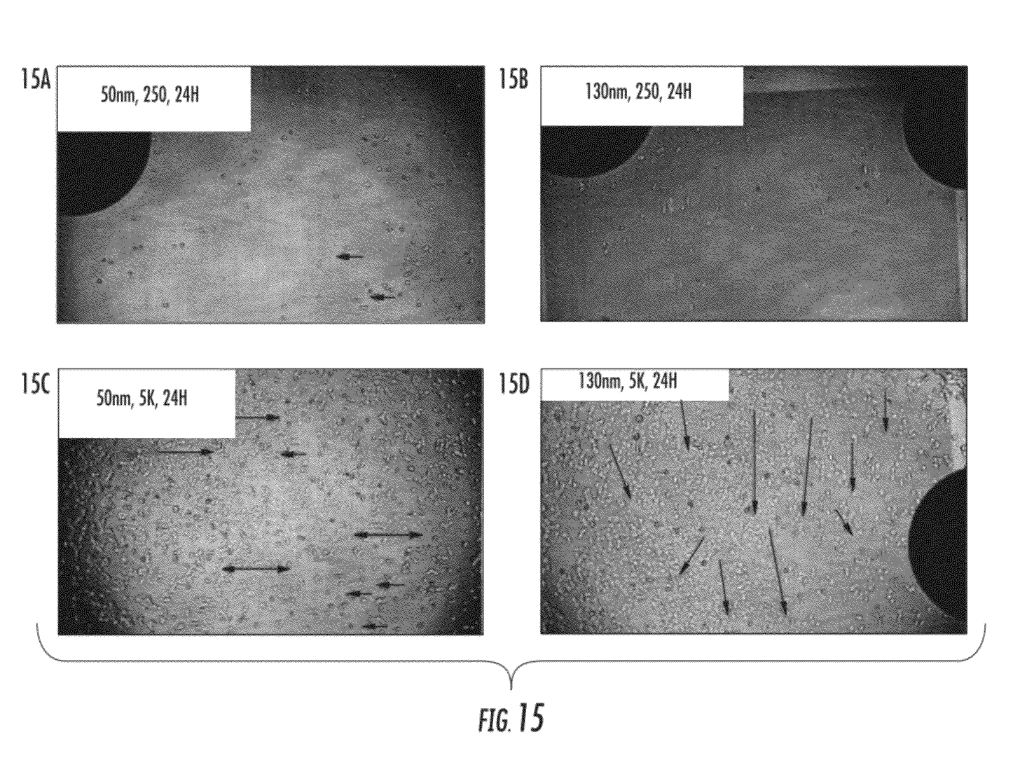

FIG. 15 shows microscope images of a comparative commercial plate (left side) and a disclosed plate (right side), each having A431 cells, seeded at 5 k cells/well and 250 k cells/well, and aligned with the grating direction for the disclosed sensors. The results demonstrate apparent preferential alignment of adhered cells for the disclosed sensors.

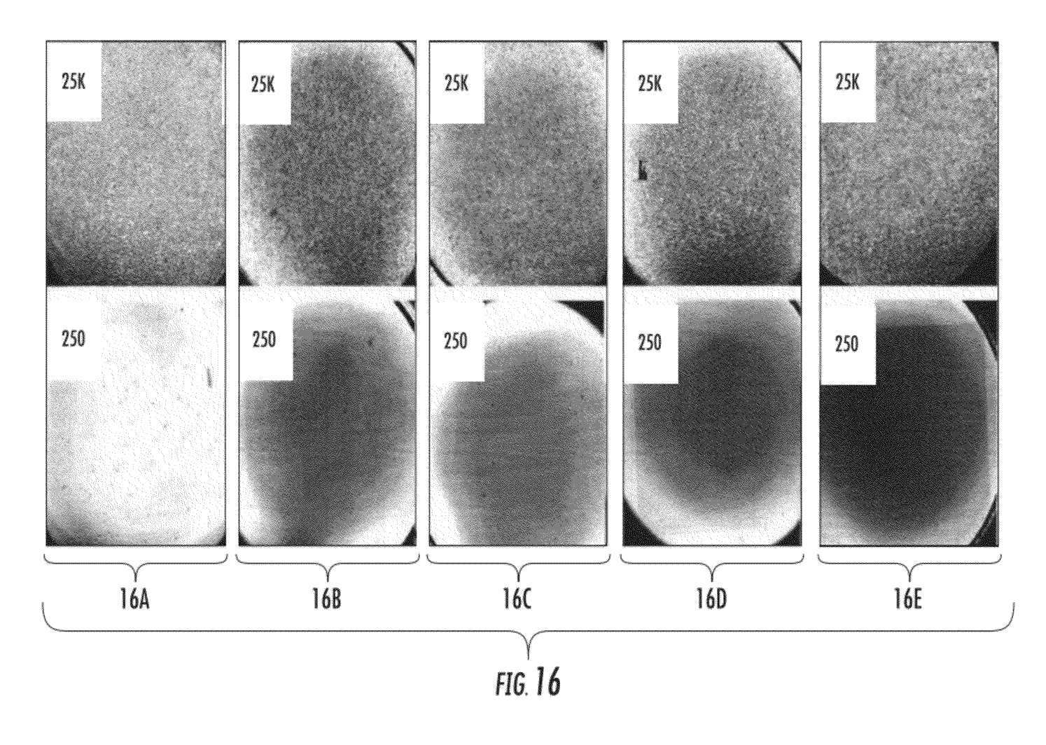

FIG. 16 shows microscope images of THP-1 cells 2h after plating on biosensors having grating depths (left to right) of A) 50 nm, B) 100 nm, C) 110 nm, D) 120 nm, and E) 130 nm. The THP-1 cells were seeded at 25 k cells/well (top series) and 250 k cells/well (bottom series).

The glass master stamps were used with the UV curable resin and replicated into several copy plates (25 plates) from which several copy plates were used for tests in deposition of niobia. Eight (8) plates were prepared having several different grating depths for tests with biological material and different class of cell based assays.

To investigate and compare the performance of the disclosed developed sensors with the commercial Epic® sensors for cell-based assay applications, three types of human cell lines, which exhibit different cell morphology and attachment properties, were tested. The three cell lines were: A549, a human lung carcinoma cell line with weakly adherent growth properties and epithelial morphology; A431, a human skin carcinoma cell line with strongly adherent growth properties and epithelial morphology; and THP-1, a human leukemic cell line with suspension cell growth properties and monocytic morphology. A549 and A431 cells were each seeded at three different cell densities so as to obtain cell confluency of ˜100%, 80%, and <1% after 24 h of culture. FIGS. 12 and 14 show that all the disclosed sensors supported the attachment and growth of both A549 and A431 cells and that the disclosed sensors and the commercial Epic® sensors showed comparable cell growth and attachment. THP-1 cells similar showed comparable attachment to the disclosed and commercial sensor surfaces (FIG. 16). Comparison of the morphology of sub-confluent A549 and 431 cells, on the other hand, showed that the morphology of the cells was sensor-dependent. For example, whereas A549 cells showed perpendicular alignment with the grating of all sensors tested (FIG. 13), A431 cells showed perpendicular alignment with the grating of commercial sensors and with disclosed sensors having grating depth of 100-110 nm. On the disclosed sensors with grating depths of 120 and 130 nm, these cells aligned with the grating direction (FIG. 15).

A series of experiments was used to confirm the theoretical model of higher spatial resolution and high sensitivity of the plates. For that a disclosed method of analysis for a typical cell assay in the experiments was performed.

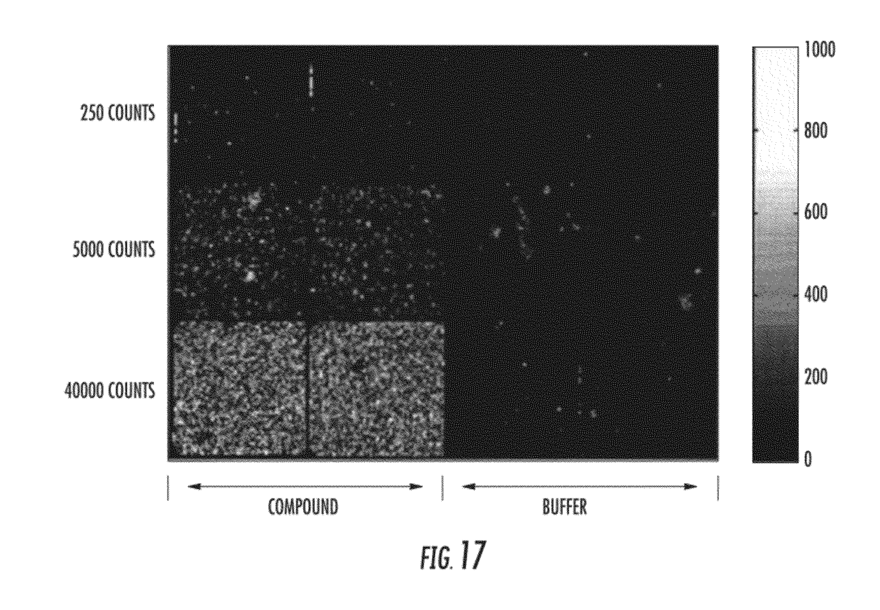

First the wells were separated with different cell count concentrations small (250 cells), medium (5000 cells) and large (40000 cells). Regions for tests with compound and buffer were also separated with repeat sections. This experimental layout is shown in FIG. 17.

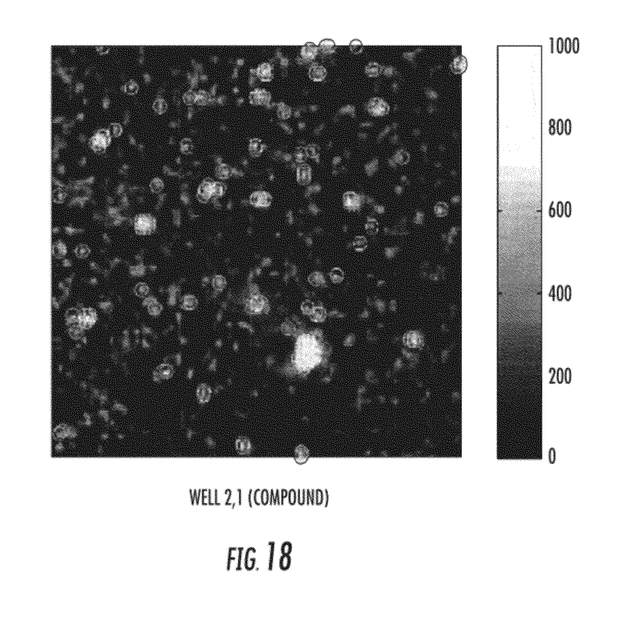

Second, to optimize the analysis and be able to detect small cell patters one performed a selection of the responders. Each pixel has attached to its position its individual time domain information. Here, we selected by software only the pixels corresponding to the maximum responders—that is, those displaying 50%-100% of the maximum response. FIG. 18 shows selections of responders. Each pixel has attached to itself its individual time domain information. The pixels corresponding to the maximum responders were selected, that is, those displaying 50% to 100% of the maximum response. This avoids processing information in space(s) (pixels) where the highest responder cells are not present.

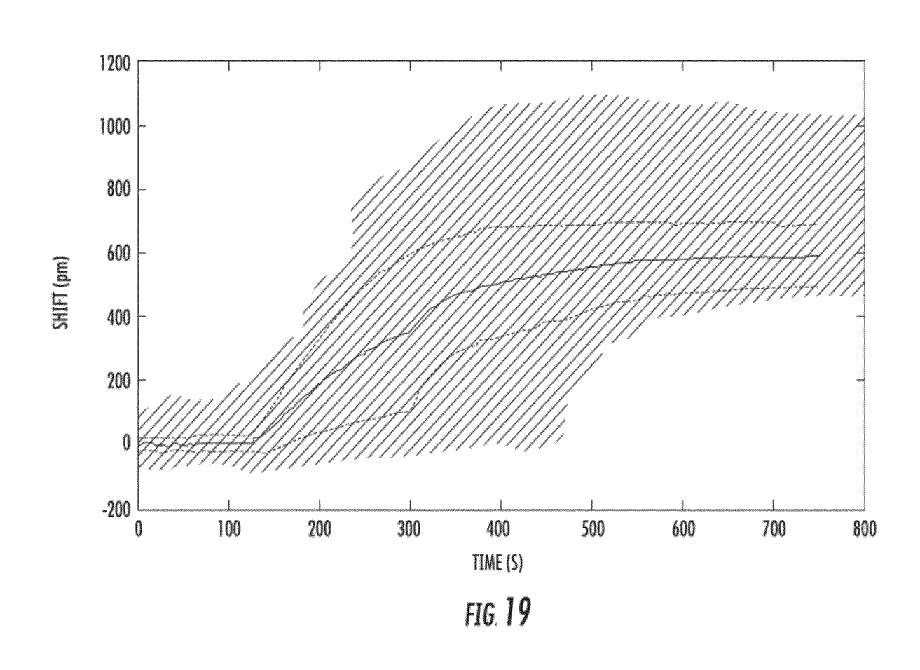

Third, the time traces and averaging of selected responders are computed. An example of this process is shown in FIG. 19. FIG. 19 shows cumulative time traces (gray) and averaged traces (three distinct single lines) for selected responders. Each pixel was then analyzed in the time domain and the ensemble average and standard deviation of all pixels selected was traced and computed leading to a final average and standard deviation of the cell response. With the information of all averages and standard deviation of all the cell densities tried one can analyze and compare the performance of the disclosed plates in contrast to the standard Epic® plate.

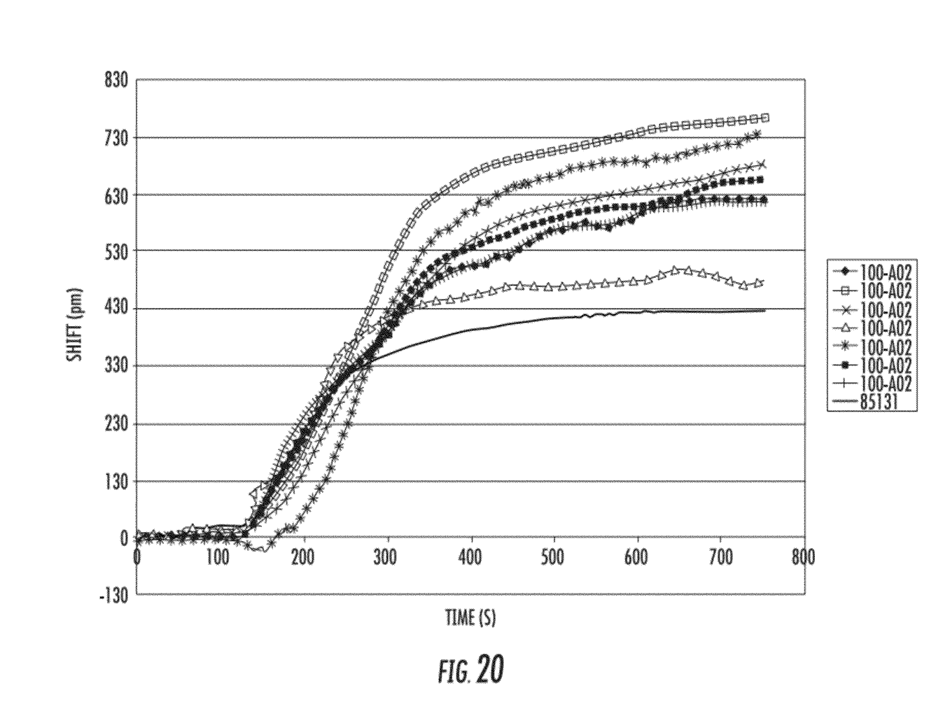

FIG. 20 provides a comparison of average traces for THP-1 cell with medium cell concentration (5 k cells) for the several different plates having various grating teeth depths (H) of 100 nm, 110 nm, 120 nm, 130 nm, and 50 nm (plate 85131). With all this information for all cell densities simple statistics of the results can be outlined. The results are restricted to cell lines A431 and THP-1 simply because they were performed in similar fashion to avoid an experimental bias.

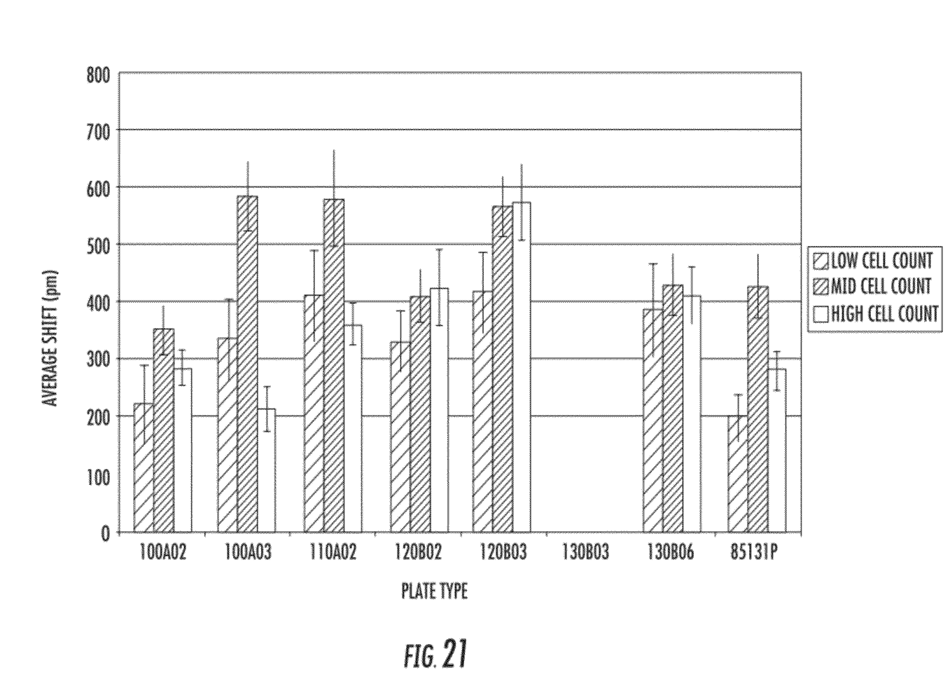

FIG. 21 shows bar chart statistics of results for tests with A431 cells that show average wavelength shifts for several different experimental plates and the error bars indicating its standard deviation. Improvements for the disclosed sensor plates are evident in contrast to plate 85131p (far right) for the low and medium cell counts. For high cell counts the improvements were minimal.

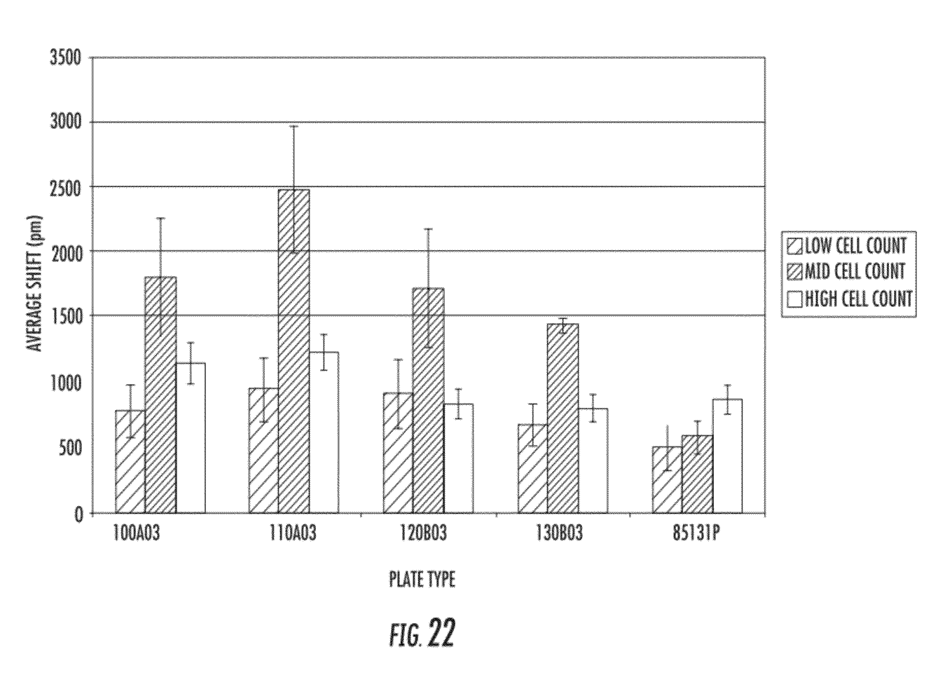

FIG. 22 shows bar chart statistics of results for tests with THP-1 cells showing average wavelength shifts for several different experimental plates and with error bars indicating its standard deviation. Improvements for the disclosed sensor plates are evident in contrast to plate 85131P for the low cell counts (reproducibility was demonstrated with two separate experiments). For medium cell counts the results were similar, although with a smaller improvement. For high cell counts the improvements were minimal. The disclosed cell assay plates show definite improvement for low and medium cell concentrations in contrast to the standard Epic® plates.

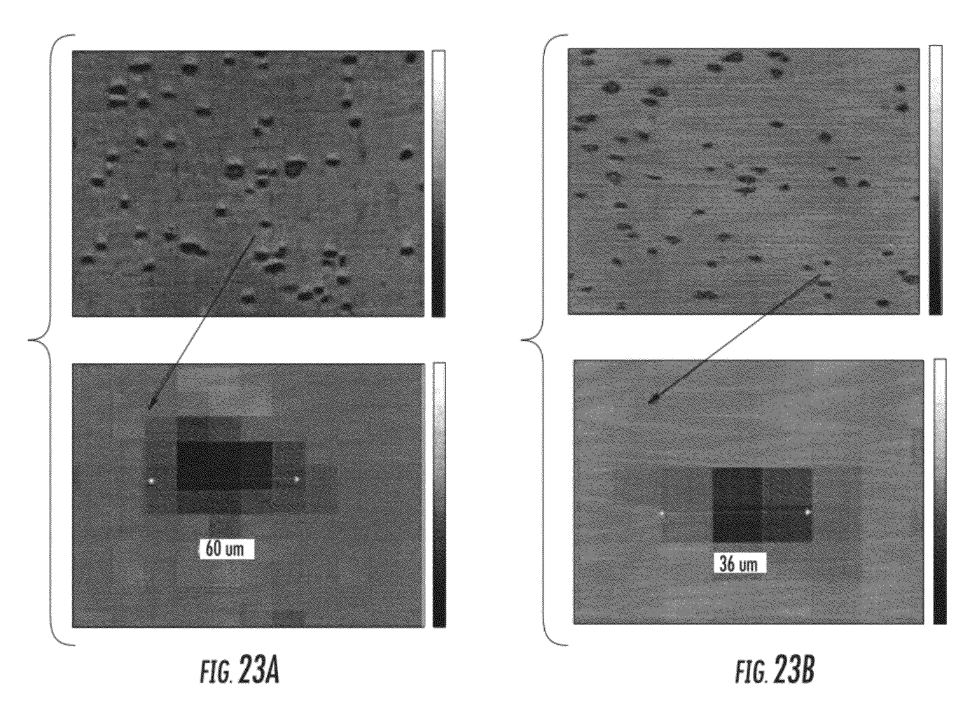

Finally, FIGS. 23a and 23b show microscopic images of the power reflectivity of, respectively, the comparative commercial standard plate (FIG. 23a) (left side) having a grating teeth height of 50 nm compared to the disclosed plate (FIG. 23b) (right side) having a grating teeth height of 120 nm. The approximate pixel resolution of the label-free detection instrument used was around 12 microns. Magnification on the smallest cells revealed a reduction of the beam width spot size for the standard plate compared to the disclosed plate. The ratio of reduction in the beam width on the sensor agrees well with the FDTD simulations performed with Gaussian beams indicating an improvement in resolution of about two times to about two and one-half times (i.e., 2× to 2.5×).

The disclosed sensor, and corresponding well plate incorporating the biosensor configuration can provide, for example: enhanced angular tolerance attributable to, for example, wider wavelength bandwidth; increased spatial resolution; and the added benefit of higher sensitivity for peak responders in low- and medium-cell concentrations.

### Spatial Resolution of Resonant Waveguide Grating Coupler (RWGC).

The Epic® sensor is a waveguide grating coupler. Resonant coupling occurs when the phase matching condition is satisfied:

$$\frac{λ}{Λ} \pm sin θ = {n}_{eff} \qquad ( 1 )$$

where θ₁ is the incident angle, λ the resonant wavelength, Λ the grating pitch, and n_{eff} the effective index of the waveguide. The plus sign represents the forward propagating leaky wave, and the negative sign for reverse propagating leaky wave. Given the grating structure and the waveguide material and thickness, the spectral profile of the resonance can be simulated using rigorous coupled wave analysis (RCWA). Simulation can be accomplished using, for example, G-Solver® (www.gsolver.com) or like diffraction grating simulation software. For Epic® sensors the grating pitch can be 500 nm, the depth can be 50 nm, and the thickness of the niobia waveguide can be 146 nm.

Various imaging methods can be used to acquire the images. These include full field imaging using for example, a 2D image sensor, raster scanning, line scanning, or like methods. The following example demonstrates the disclosed high resolution and high sensitivity article and methods.

The disclosure has been described with reference to various specific embodiments and techniques. However, it should be understood that many variations and modifications are possible while remaining within the scope of the disclosure.

1. An optical sensor comprising:
a substrate;
a waveguide grating adjacent the substrate; and
a waveguide coat layer adjacent the waveguide grating,
the waveguide coat layer having a thickness (W) of from 30 nm to 300 nm, and
the waveguide grating having a teeth height (H) of from 0.2×W to 1×W.

2. The sensor of claim 1 wherein the waveguide coat layer thickness (W) is from about 135 nm to about 160 nm, and the waveguide grating teeth height (H) is from 100 nm to 150 nm.

3. The sensor of claim 1 wherein the waveguide core thickness (W_{core}=W−H) is from 5 nm to 50 nm.

4. The sensor of claim 1 wherein the resolution is increased by from 2 to 3 times and the angular sensitivity is decreased by from 1.1 to 2.5 times compared to a sensor having a waveguide grating having a teeth height (H) of 50 nm.

5. The sensor of claim 1 wherein the common waveguide core thickness of consecutive grating teeth, W_{core}=(W−H), that connects the grating W and H dimensions is between 0 nm to 110 nm.

6. The sensor of claim 1 wherein the common waveguide core thickness of consecutive grating teeth, W_{core}=(W−H), that connects the grating W and H dimensions is from −50 nm to 50 nm.

7. The sensor of claim 1 wherein the index of refraction of the waveguide material is from 1.6 to 3.4.

8. The sensor of claim 7 wherein the index of refraction of the waveguide coat material is from about 2.0 to about 2.4 and the waveguide coat material is niobia.

9. The sensor of claim 1 wherein the substrate comprises at least one of a polymer, a composite, a metal, a glass, an inorganic oxide, an inorganic nitride, or a combination thereof, having an index of from 1.3 to 2.2.

10. The sensor of claim 1 wherein the substrate, waveguide grating, and waveguide coat layer has a low-loss in the wavelength of operation and having optical power attenuation at the wavelength of operation of less than or equal to 3 db/cm.

11. The sensor of claim 10 wherein the wavelength of operation is from 200 nm to 2,000 nm.

12. The sensor of claim 1, wherein the substrate comprises a glass, a plastic, or a combination thereof, the waveguide grating comprises a glass, and the waveguide coat layer comprising Ta₂O₅, Nb₂O₅, TiO₂, Al₂O₃, SiO₂, silicon nitride, or a mixture thereof, wherein the waveguide coat layer is adjacent to the surface of the substrate.

13. The sensor of claim 1, wherein the sensor is a biosensor.

14. The sensor of claim 1, wherein the sensor is a resonant waveguide grating sensor.

15. A microplate having at least one sensor of claim 1.

16. A system for label-free detection of an analyte in a microplate, the system comprising:
a light source for illuminating the at least one sensor of a microplate;
a receptacle to receive the microplate of claim 15; and
an imager to receive the optical image of the at least one sensor of the microplate.

17. The system of claim 16 wherein the imager has a pixel size of about 0.1 to 100 micrometers.

18. A method of using the sensor of claim 1 comprising:
depositing at least one live-cell on the surface of the sensor; and
interrogating the sensor with a suitable reader having a radiation source.

19. The method of claim 18 wherein the at least one live-cell on the surface of at least one sensor comprises from two to about 500 live-cells.

20. The method of claim 18 wherein the depositing at least one live-cell on the surface of the sensor produces preferential alignment of the cells on the surface of the sensor with respect to the waveguide grating, the waveguide grating coat layer, an optional waveguide grating surface coat layer, or a combination thereof.
